(19) United States
(12) Patent Application Publication
(10) Pub. No.: US 2008/0269338 A1
(43) Pub. Date: Oct. 30, 2008

(54) AQUEOUS SOLUTIONS CONTAINING CHLORAMINE WHICH ARE FREE FROM DI-AND TRICHLOROAMINE, AS WELL AS FROM AMMONIA

(76) Inventors: Waldemar Gottardi, Innsbruck (AT); Barbara Teuchner, Innsbruck (AT); Markus Nagl, Axams (AT)

Correspondence Address: PROSKAUER ROSE LLP;PATENT DEPARTMENT, 1585 BROADWAY, NEW YORK, NY, 10036-8299, US

(21) Appl. No.: 11/742,303

(22) Filed: Apr. 30, 2007

(51) Int. Cl. A61K 31/185; A61P 29/00; A61P 31/04

(52) U.S. Cl. 514/578

(57) ABSTRACT

The combination of N-chlorotaurine, preferably in the form of sodium salt, and ammonium chloride in aqueous solution absent a buffer is a powerful antiseptic preparation sufficiently stable for use in topical treatment of infections, inflammation and oozing tissue deficiencies. The outstanding microbicidal properties of the preparation are founded in the formation of monochloramine, which is distinguished by the absence, or substantial absence, of dichloramine and trichloramine.

This invention relates to stable aqueous solutions of monochloramine (NH₂Cl) procedures for their preparation, and the use of such solutions in the treatment of microbial infections, inflammations, and oozing tissue deficiencies.

### BACKGROUND OF THE INVENTION

Monochloramine (NH₂Cl) exhibits, like all active chlorine compounds, oxidizing and bactericidal properties.

Because of its instability, NH₂Cl cannot be stored under normal conditions (it disproportionates to dichloroamine, which decomposes to ammonia, chloride and nitrogen) and has to be prepared in situ. Valentine et al., “General Acid Catalysis of Monochloramine Disproportionation,” ENVIRON. SCI. TECHNOL. 1988; 22: 691-696. It is used, for example, in water disinfection (drinking, sewage, cooling waters) and can be made from ammonia or ammonium salts and elemental chlorine or active chlorine compounds (bearing O—Cl or N—Cl functions).

In biological systems, NH₂Cl is formed during the reaction of organic chloramines (produced by stimulated granulocytes) with ammonium. Grisham et al., “Chlorination of Endogenous Amines by Isolated Neutrophils,” J. BIOL. CHEM. 1984; 259: 10404-10413. Monochloramine exhibits a very high bactericidal power, which is likely attributable to its lipophilic nature, making penetration into bacteria easier. In addition, it is known that NH₂Cl penetrates human tissue markedly better than another endogenous antibacterial agent, N-chlorotaurine (NCT).

NH₂Cl is one of the reaction products of NCT and ammonium chloride. An in-vitro study of bactericidal efficacy was done with freshly prepared, phosphate-buffered solutions of NCT and ammonium chloride. Compared to plain NCT solution, the solutions of NCT and ammonium chloride showed a significant increase in bactericidal power and presented impressive results against important pathogens like mycobacteria and fungi. Nagl et al., “Enhancement of the Bactericidal Efficacy of N-Chlorotaurine by Inflammation Samples and Selected N—H Compounds,” HYG. MED. 1996; 21: 597-605; Nagl et al., “Rapid Killing of Mycobacterium terrae by N-Chlorotaurine in the Presence of Ammonium is Caused by the Reaction Product Monochloramine,” J. PHARM. PHARMACOL. 1998; 50: 1317-1320; Nagl et al., “Enhanced Fungicidal Activity of N-Chlorotaurine in Nasal Secretion,” J. ANTIMICROB. CHEMOTHER. 2001; 47: 871-874. These references are incorporated in their entirety herein by reference.

NH₂Cl is formed in the reaction of ammonia or ammonium salts and other active chlorine compounds, e.g., hypochlorites, chloroisocyanuric acids, and N-chloro-4-toluenesulfonamide sodium (chloramine T). However, in general these reactions do not stop after mono-chlorination and produce also di- and trichloroamine (NHCl₂ and NCl₃). Grisham et al., supra; Thomas et al., “Preparation and Characterization of Chloramines,” METHODS ENZYMOL. 1986; 132: 569-585.

The formation of NHCl₂ and NCl₃ is not wanted because these compounds are, contrary to NH₂Cl, extremely unpleasant, pungent smelling compounds whose presence would exclude use of such a disinfectant in practice.

The preparation of aqueous solutions containing only monochloramine requires special conditions, mainly an alkaline environment of about pH 9 and a molar surplus of ammonia. Beck et al., “Preformed Monochloramine Used as a Post-Disinfectant in Drinking Water Treatment at Sjaelsoe Water Works,” AQUA 1986; 1: 25-33; Rizk-Ouaini et al., “Oxidation reaction of ammonia with sodium hypochlorite: Production and degradation reactions of chloramines,” BULLETIN DE LA SOCIETE CHIMIQUE DE FRANCE 1986; 4: 512-21.

Thus, the preparation of monochloramine solutions with an alkaline pH would prevent the formation of unwanted NHCl₂ and NCl₃. However, the reaction produces ammonia, which is undesirable because of its unpleasant odor.

A monochloramine-containing preparation for use as an antiseptic in human medicine ideally would have a pH of about 7-8 and be free from ammonia, NHCl₂ and NCl₃. Possible candidates include aqueous solutions of an ammonium salt (e.g. ammonium chloride) and a stable active chlorine compound such as calcium hypochlorite, sodium dichloro-isocyanurate, or chloramine-T. Sodium hypochlorite is not suitable because of its instability. A suitable package might consist of two separated compartments, one containing the ammonium salt and the other containing the active chlorine compound. The active solution could be made immediately before use by dissolving both substances in water:

NH₄⁺+H₂O [symbol] NH₃+H₃O⁺

R—Cl+NH₃→R—H+NH₂Cl

R=>—N—, —O—

However, when chloramine-T is used as the chlorinating agent (a likely choice given its excellent stability in aqueous solution) the reaction does not stop at NH₂Cl. Even in the presence of excess ammonium, the higher chlorinated derivatives, NHCl₂ and NCl₃, are formed and the pH drops from 6.8 to 2.9 within 8 minutes. The underlying reactions are as follows:

2NH₂Cl [symbol] NH₃+NHCl₂ (disproportionation)

NHCl₂+NH₂Cl [symbol] NCl₃+NH₃ (disproportionation)

NHCl₂+NH₂Cl→N₂+3H⁺+3Cl⁻ (redox reaction)

If the same experiment is done in the presence of 0.1 M phosphate buffer, the pH drops from 7.0 to only 6.3 within 2 hours. However, decomposition under liberation of nitrogen gas and a 90% loss of oxidation capacity occurs within 1 hour. Calcium hypochlorite and sodium dichloro-isocyanurate behave in the same manner.

The chemical nature and the stability of NCT and ammonium chloride were investigated and reported in Gottardi et al., “N-Chlorotaurine and ammonium chloride: An antiseptic preparation with strong bactericidal activity,” Int. J. Pharmaceut. (2006) doi:10.1016/j.ijpharm.2006.11.003. This reference is incorporated in its entirety herein by reference. Surprisingly, it was found that when NCT is combined with ammonium salts in plain water in the absence of a buffer, the reaction goes only to the stage of NH₂Cl. Unwanted by-products NHCl₂ and NCl₃ are not formed. As a result of the lack of a buffer in this procedure, the initial pH showed no defined value but ranged within about 6.0 to about 8.0 if the concentrations of each of NCT and ammonium chloride were between about 0.01% and about 1.0% w/v. In this pH range, liberation of appreciable amounts of ammonia did not take place. This pH range is compatible with use in medical practice. Additionally, the composition has no buffering potency and therefore adapts to the prevailing buffered system. Finally, the solutions exhibited unexpected stability.

### SUMMARY OF THE INVENTION

In one aspect, the invention is a composition comprising an aqueous solution of NCT and an ammonium salt that is free, or substantially free, of unwanted byproducts NHCl₂ and NCl₃. The phrase “substantially free” means that the aqueous solution of NCT and an ammonium salt contains less than 10⁻⁵ mol/L NHCl₂ and NCl₃.

In another aspect, the invention is an aqueous solution of NCT and an ammonium salt at a pH of about 6.0 to about 8.0 in the absence of a buffer.

In yet another aspect, the invention is an aqueous solution of NCT and an ammonium salt, absent a buffer, and free (or substantially free) of NHCl₂, NCl₃ and ammonia.

In yet another aspect, the invention is an aqueous solution of NCT and an ammonium salt, absent a buffer, having a pH of about 6.0 to about 8.0, and free (or substantially free) of NHCl₂, NCl₃ and ammonia.

In another aspect, the invention is a procedure for preparing an aqueous solution of NH₂Cl comprising the steps of reacting N-chlorotaurine, preferably a salt of N-chlorotaurine, with an ammonium salt in aqueous solution, wherein the procedure is performed at a pH of about 6.0 to about 8.0 and in the absence of a buffer. The resulting product of the procedure is free of NHCl₂, NCl₃ and ammonia.

In another aspect, the invention is a method for the treatment of microbial infections, inflammations, and oozing tissue deficiencies comprising administering to a patient in need of such treatment a pharmaceutically acceptable amount of an aqueous solution of NCT and an ammonium salt.

### BRIEF DESCRIPTION OF THE DRAWINGS

FIG. 1 is a diagrammatic representation of the influence of buffer concentration on the stability of an aqueous solution of NCT and ammonium chloride upon storage at 2-4° C.

FIG. 2 is a diagrammatic representation of equilibrium concentrations of NH₂Cl in aqueous solutions of NCT and ammonium chloride.

FIG. 3 is a diagrammatic representation of the results of a kill test involving S. aureus and NCT, NCT+ammonium, and a negative control.

FIG. 4 is a series of diagrammatic representations of kill tests involving six bacterial strains, NCT+ammonium and a negative control.

### DETAILED DESCRIPTION

As a source of NCT, the sodium salt, NCT-Na, is preferable. The ammonium salt may be any non-toxic ammonium salt. Because of the ubiquitous presence of chloride in the human body, ammonium chloride is preferred.

Preparations of antiseptic NCT-Na and ammonium chloride may be made by dissolving the two ingredients in water without a buffer and stored in a refrigerator. Under these conditions, the solutions will maintain an adequate antiseptic activity for at least one month.

In general, the concentrations of NCT and the ammonium salt may each be in the range of about 0.01% to about 10.0% (w/v). Preferably, the concentration of NCT will be in the range of about 0.1% to about 1.0% w/v (5.5E-3 to 5.5E-2M) and the concentration of ammonium chloride will be in the range of about 0.01% to about 0.1% w/v (1.87E-3 to 1.87E-2M).

As a result of the lack of a buffer, the initial pH will range within about 6.0 to about 8.0 if the concentrations of each of NCT and ammonium chloride are between about 0.01% and about 1.0%. In this pH range, liberation of appreciable amounts of ammonia do not take place. This pH range is compatible with use in medical practice. Additionally, the composition has no buffering potency and therefore adapts to the prevailing buffered system.

The initial pH of the inventive solutions changes with time. 1% NCT (0.055 M) in the presence of 0.01, 0.1 and 1.0 M ammonium chloride showed an initial pH of 7.92, 7.03, and 6.19, respectively. In case of 0.01 M ammonium chloride, the pH dropped within 30 minutes to 7.81, while it increased in 0.1 and 1.0 M ammonium chloride to 7.20 and 6.58, respectively. Long-term (more than 2 months) equilibrium pH values at 1-3° C. are shown in Table 1. They disclose a change of only +0.2, +0.3, and −0.3 pH units for 1% NCT in the presence of 0.02%, 0.1% and 0.5% ammonium chloride, respectively.

TABLE 1
Change of pH, c(Ox), and NH₂Cl capacity (assessed by vacuum
distillation) of 1% NCT solutions spiked with 0.02%, 0.10% and 0.5% ammonium chloride after
a period of 2 months in the refrigerator (1-3° C.)

| | | | | | | | | | |
|---|---|---|---|---|---|---|---|---|---|
| c(NH₄Cl) | | 0.02% | | | 0.1% | | | 0.5% | |
| Age of solution | 0 | | 67 | 0 | | 67 | 0 | | 67 |
| (days) | | | | | | | | | |
| pH | 8.10 | | 8.30 | 7.70 | | 8.05 | 7.30 | | 7.61 |
| c(Ox)(mol/L) | 5.50E−2 | | 5.36E−2 | 5.50E−2 | | 4.93E−2 | 5.50E−2 | | 4.30E−2 |
| [symbol] | | −2.5% | | | −10.3% | | | −21.8% | |
| NH₂Cl- | 0.70 | | 0.25 | 2.24 | | 0.98 | 8.08 | | 2.01 |
| capacity | | | | | | | | | |
| (μmol/mL) | | | | | | | | | |
| [symbol] | | −64.3% | | | −56.3% | | | −75.1% | |

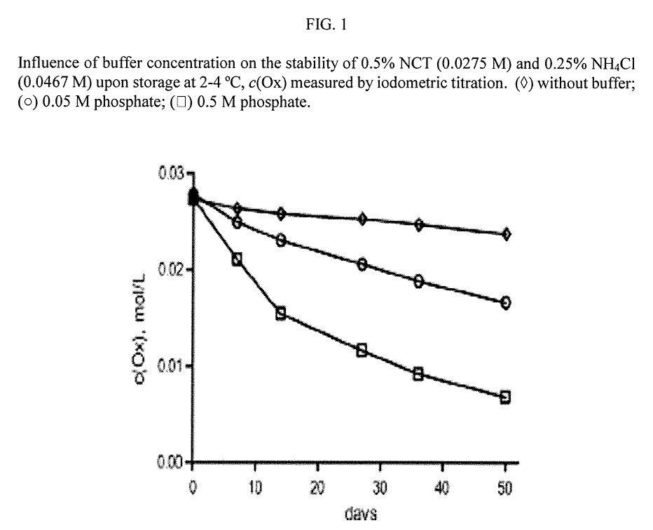

The advantage of formulating without a buffer is demonstrated in FIG. 1, which shows the influence of buffer concentration on the stability of 0.5% NCT (0.0275 M) and 0.25% NH₄Cl (0.0467M) upon storage at 2-4° C. As noted in FIG. 1, phosphate buffer contributes to the loss of c(Ox), which phenomenon correlates with buffer concentration. Iodometric titrations were performed with 0.100 M thiosulfate at pH 2-3 (acetic acid) using the automatic titration assembly TIM900 from Radiometer, Copenhagen.

The inventive solutions exhibit a decrease of only about 1.4% oxidation capacity per day when stored in the refrigerator (0-3° C). Accordingly, the stability is sufficient for 10-15 days medical attention. Solutions stored in this way are free, or substantially free, of the decomposition products NHCl₂ and NCl₃. In monochloramine solutions free of N-chlorotaurine, however, a complete conversion to dichloramine takes place under the same conditions (see Example 2).

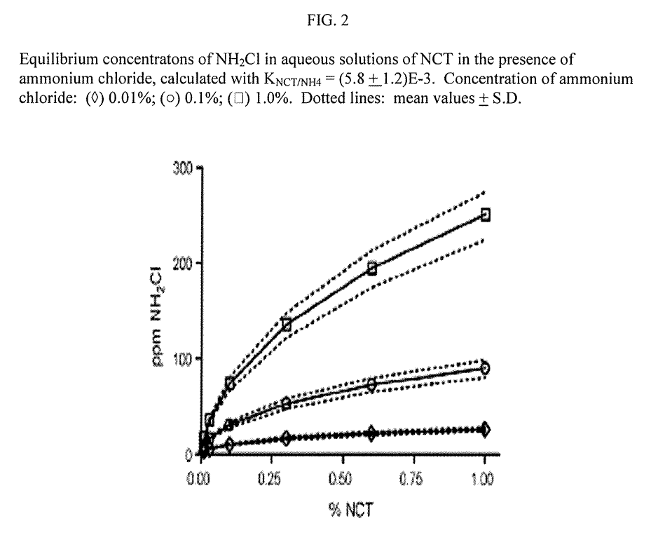

The disinfecting power of the inventive solutions may be tailored to the desired site of application and/or kind of microorganism. FIG. 2 demonstrates that combinations of NCT and ammonium chloride, each in the range of 0.01-1.0%, are capable of providing equilibrium concentrations of 5-254 ppm, the upper limit representing a very powerful formulation. (See also Table 2)

TABLE 2
Initial equilibrium concentrations of NCT and NH₂Cl in formulations
suggested for practice (calculated with K_{NCT/NH4} = 5.8E−3).

| c(NCT) | c(NH₄Cl) | [NH₂Cl] | [NCT] | [NH₂Cl] | |
|---|---|---|---|---|---|
| (%) | (%) | (ppm) | (mol/L) | (mol/L) | [NCT]/[NH₂Cl] |
| 1.0 | 1.0 | 254 | 5.0E−2 | 4.9E−3 | 10 |
| 1.0 | 0.5 | 192 | 5.1E−2 | 3.7E−3 | 14 |
| 1.0 | 0.1 | 91 | 5.3E−2 | 1.8E−3 | 30 |
| 1.0 | 0.01 | 27 | 5.5E−2 | 5.2E−4 | 105 |
| 0.1 | 0.01 | 11 | 5.3E−3 | 2.1E−4 | 26 |
| 0.01 | 0.01 | 4.8 | 4.8E−4 | 6.7E−5 | 7 |

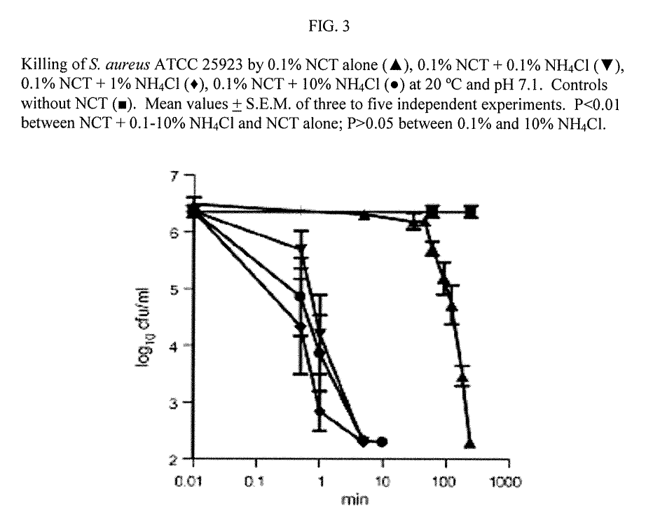

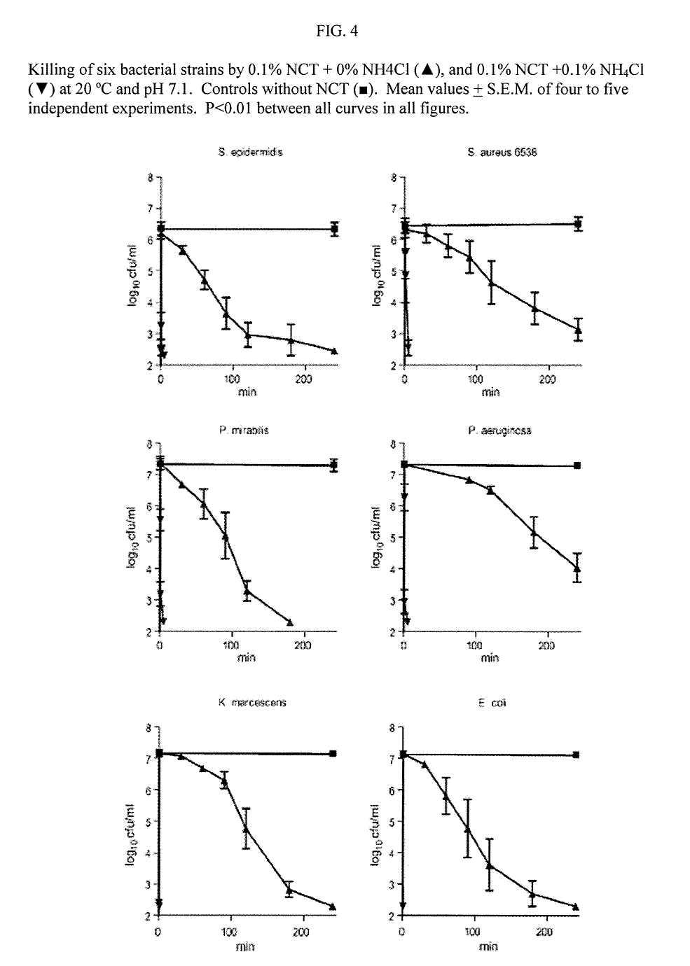

Microbicidal activity. Compared to plain NCT without additives, NCT plus ammonium chloride demonstrates enhanced bactericidal activity, about 200-300 fold. A solution of 0.1% NCT plus 0.1% ammonium chloride reduced the viability of S. aureus ATCC 25923 significantly within 1 minute, and no more viable bacteria could be detected after 5 minutes (FIG. 3; detection limit 200 CFU/mL which equals 2.30 log₁₀). Further elevation of the ammonium chloride concentrations (FIG. 3) and the NCT concentration did not lead to significantly more rapid killing. The impact of ammonium chloride on the microbicidal activity of NCT could be confirmed in several bacterial strains (FIG. 4).

Antiseptic preparations. Aqueous solutions of NCT and ammonium chloride, as described herein, provide a highly effective and well tolerable antiseptic preparation appropriate to a treatment cycle of at least 1 month if stored in the refrigerator.

Prior experiences with plain NCT solutions proved 1% and 0.1% as suitable concentrations in several clinical studies (Nagl el al., “Tolerance of N-chlorotaurine, a new antimicrobial agent, in infectious conjunctivitis—a phase II pilot study,” OPHTHALMOLOGICA 214, 111-114 (2000); Nagl et al., “Tolerability and efficacy of N-chlorotaurine compared to chloramine T for treatment of chronic leg ulcers with purulent coating,” BR. J. DERMATOL. 149, 590-597 (2003); Neher et al., “Tolerability of N-chlorotaurine in chronic rhinosinusitis applied via yamik catheter,” AURIS NASUS LARYNX 32, 359-364 (2005); Neher et al., “Acute otitis externa: efficacy and tolerability of N-chlorotaurine, a novel endogenous antiseptic agent,” LARYNGOSCOPE 114, 850-854 (2004); Romanowski et al., “N-Chlorotaurine is an effective antiviral agent against adenovirus in vitro and in the Ad5/NZW rabbit ocular model,” INVEST. OPHTH. VIS. SCI. 47, 2021-2026 (2006); Teuchner et al., “Tolerability and efficacy of N-chlorotaurine in epidemic keratoconjunctivitis—a double-blind randomized phase 2 clinical trial, J. OCULAR PHARMACOL. THER. 21, 157-165 (2005). They should be the starting concentrations for NCT/ammonium chloride formulations which are completed with an appropriate concentration of ammonium chloride (see Table 1). NCT concentrations lower than 0.1% are not recommended due to insufficient oxidation capacity. Since disinfection processes with active halogen agents are always associated with consumption (i.e. reduction) of c(Ox), an adequate reservoir of NCT enables the formation of NH₂Cl, which, because of its higher reactivity, will be used up first of all.

In vivo studies with plain NCT show good tolerability in the rabbit and human eye (Nagl et al., “Tolerance of N-chlorotaurine, an endogenous antimicrobial agent, in the rabbit and human eye—a phase 1 clinical study,” J. OCULAR PHARMACOL. THER. 14, 283-290 (1998); Nagl et al., 2000, supra). In vivo tests with mixtures of 1% NCT and 0.1% ammonium chloride, yielding [NH₂Cl] of about 91 ppm revealed good tolerance and efficacy in adenoviral conjunctivitis in the rabbit eye (Romanowski et al., 2006). On normal skin, higher concentrations are possible. A patient suffering from a fungal infection was treated with a 1%/1% mixture (254 ppm NH₂Cl), which caused marked burning. However, healing was achieved within only 2 days.

The invention is further explicated by means of the following examples.

### EXAMPLE 1

### Preparation and Stability of an Aqueous NH₂Cl-Containing Solution made from 1% N-Chlorotaurine and 1% Ammonium Chloride

Each 1 g N-chlorotaurine and ammonium chloride were dissolved in 100 mL water, at which a pH 7.5 was settled. After storing 10 days in the refrigerator (0-3° C.) or at room temperature, a pH of 7.5 or 7.1 was measured. The oxidation capacity, assessed by iodometric titration, decreased within the same period from 5.43e-02 M Cl⁺ to 4.82e-02 M Cl⁺ (3.35e-02 M Cl⁺) which equals a daily decrease of 1.1% (3.8%).

The concentration of NH₂Cl was measured photometrically using the known UV spectra of N-chlorotaurine and NH₂Cl. Gottardi et al., “Chemical Properties of N-Chlorotaurine Sodium, a Key Compound in the Human Defence System,” Arch. Pharm. (Weinheim) 2002; 335: 411-421; Snyder et al., “Kinetics of Chlorine Transfer from Chloramine to Amines, Amino Acids, and Peptides,” Inorg. Chem. 1982; 21: 2545-2550. The initial concentration was [NH₂Cl]=3.88e-03 M (205 ppm) and decreased within 10 days at 0-3° C. to 191 ppm or 158 ppm if stored at room temperature.

### EXAMPLE 2

### Preparation and Stability of a Pure Monochloramine Solution

A solution of 1 g each of N-chlorotaurine and ammonium chloride in 100 mL water was distilled in a rotavapor (water stream vacuum, water bath temperature 50° C.). The distillate contained, according to the UV spectrum, 2.13e-3 M pure monochloramine (NH₂Cl, absorption band at λₘₐₓ=244 nm, A₂₄₄=0.9812, d=0.1 cm, ε₂₄₄=461.6 L mol⁻¹ cm⁻¹). After three days storage in the refrigerator at 0-3° C., the band at 244 disappeared, while two bands at 203 nm (A=1.146) and 294 nm (A0 0.148) appeared which are characteristic for the chromophor —NCl₂. Gottardi et al., supra. These spectral changes indicate a complete conversion of NH₂Cl to NHCl₂.

### EXAMPLE 3

### Preparation of an Isotonic NH₂Cl Containing Solution made from 1% N-Chlorotaurine and 0.53% Ammonium Chloride

1 g N-chlorotaurine (0.0055 mol) and 0.53 g ammonium chloride (0.0099 mol) were dissolved in 100 mL water. The total molarity came then to 0.154 mol/L, which corresponds with an isotonic 0.9% sodium chloride solution. The pH was 7.2, and its chloramine concentration came to [NH₂Cl]=2.48e-03 M or 128 ppm.

### EXAMPLE 4

### Temporal Course of the Bactericidal Efficacy of an NH₂Cl-Containing Solution made of 1% N-Chlorotaurine and 1% Ammonium Chloride

An aqueous solution of 1% N-chlorotaurine and 1% ammonium chloride was stored in the refrigerator (0-3° C.) for 8 days. After 8 days, an additional fresh aqueous solution of 1% N-chlorotaurine and 1% ammonium chloride was prepared.

Twenty minutes later, Staphylococcus aureus was added in a final concentration of 1×10⁷ colony forming units per ml to both solutions and incubated at room temperature. After different incubation times, aliquots were removed and the number of surviving bacteria was determined by quantitative cultures. In both solutions, the bacteria were killed partially after 30 seconds and completely after 1 minute. Therefore, an aqueous solution of 1% NCT and 1% ammonium chloride exerts full bactericidal activity even after storage for 8 days in a refrigerator.

### EXAMPLE 5

### Penetration of NH₂Cl through the Human Cornea

A human cornea was clamped in a specially designed chamber. The upper compartment was filled with the disinfectant, while the lower one contained the buffer solution. The system was equilibrated for 2, 4, and 6 hours at room temperature, and afterwards the oxidation capacity was determined in the buffer solution of the lower compartment. Using 1% N-chlorotaurine, the diffused oxidation capacity was 1.3% after 2 hours, 4.8% after 4 hours, and 25.4% after 6 hours from the starting value in the upper compartment. The corresponding values for a solution of 1% N-chlorotaurine and 1% ammonium chloride were 17.9% after 2 hours, 30.1% after 4 hours, and 53.1% after 6 hours.

### EXAMPLE 6

### Microbiological Experiments

Bacterial strains, Staphylococcus aureus ATCC 25923 and 6538, Staphylococcus epidermidis ATCC 12228, Escherichia coli ATCC 11229, Proteus mirabilis ATCC 14153, Pseudomonas aeruginosa ATCC 27853, and a clinical isolate of Klebsiella marcescens deep-frozen for storage were grown overnight on tryptic soy agar (Merck, Darmstadt, Germany). Colonies from this agar were grown in tryptic soy broth (Merck, Darmstadt, Germany) at 37° C. overnight and washed twice with saline.

Killing tests. Bacteria were diluted in buffered test solution to concentrations of 1×10⁶ to 5×10⁷ CFU/mL. Immediately and subsequent to different incubation times at room temperature, aliquots were removed, and NCT with or without ammonium chloride or plain monochloramine was inactivated by 10-fold dilution in 0.3% sodium thiosulfate. Aliquots (50 μL) of these solutions as well as of 100-fold further dilutions in saline were spread onto tryptic soy agar plates with an automatic spiral plater (Don Whitley Scientific Limited, West Yorkshire, UK) in duplicates allowing a detection limit of 200 CFU/mL. Plates were grown at 37° C., and CFU were counted after 24 and 48 hours. Controls without NCT were treated the same way. Ammonium chloride as well as NCT or NCT plus ammonium chloride inactivated with sodium thiosulfate before the addition of bacteria had no influence on viability as shown in preliminary experiments.

The foregoing examples are illustrative only and are not meant to limit the scope of the inventions claimed herein.

1. A composition comprising an aqueous solution of N-chlorotaurine and an ammonium salt, wherein said composition is free, or substantially free, of unwanted byproducts NHCl₂ and NCl₃.

2. The composition of claim 1, wherein the aqueous solution contains no buffer.

3. The composition of claim 1, wherein the ammonium salt is ammonium chloride.

4. The composition of claim 1, wherein N-chlorotaurine is in the form of an alkali salt of N-chlorotaurine.

5. The composition of claim 4, wherein N-chlorotaurine is in the form of the sodium salt of N-chlorotaurine.

6. The composition of claim 1, wherein N-chlorotaurine and the ammonium salt each have a concentration in the range of about 0.01 to about 10.0% (w/v).

7. The composition of claim 6, wherein N-chlorotaurine and the ammonium salt each have a concentration in the range of about 0.01% to about 1.0%.

8. The composition of claim 7, wherein N-chlorotaurine has a concentration of about 0.1% to about 1.0% and the ammonium salt has a concentration of about 0.01% to about 1.0%.

9. A composition comprising an aqueous solution of N-chlorotaurine and an ammonium salt having no buffer and having an initial pH upon formulation of about 6.0 to about 8.0.

10. The composition of claim 9, wherein the composition is free, or substantially free, of NHCl₂, NCl₃ and ammonia.

11. The composition of claim 9, wherein the ammonium salt is ammonium chloride.

12. The composition of claim 9, wherein N-chlorotaurine is in the form of an alkali salt of N-chlorotaurine.

13. The composition of claim 12, wherein N-chlorotaurine is in the form of the sodium salt of N-chlorotaurine.

14. The composition of claim 9, wherein N-chlorotaurine and the ammonium salt each have a concentration in the range of about 0.01% to about 1.0% (w/v).

15. The composition of claim 14, wherein N-chlorotaurine has a concentration of about 0.1% to about 1.0% and the ammonium salt has a concentration of about 0.01% to about 1.0%.

16. A process for preparing an aqueous solution of NH₂Cl comprising the steps of combining N-chlorotaurine with an ammonium salt in water, wherein the process is performed at a pH of about 6.0 to about 8.0 and in the absence of a buffer.

17. The process of claim 16, wherein the aqueous solution of NH₂Cl is free, or substantially free, of NHCl₂, NCl₃ and ammonia.

18. The process of claim 16, wherein the ammonium salt is ammonium chloride.

19. The process of claim 16, wherein N-chlorotaurine is in the form of an alkali salt of N-chlorotaurine.

20. The process of claim 19, wherein N-chlorotaurine is in the form of the sodium salt of N-chlorotaurine.

21. The process of claim 16, wherein N-chlorotaurine and the ammonium salt each have a concentration in the range of about 0.01 to about 1.0% (w/v).

22. The process of claim 21, wherein N-chlorotaurine has a concentration of about 0.1% to about 1.0% and the ammonium salt has a concentration of about 0.01% to about 1.0%.

23. A method for the treatment of microbial infections, inflammations, and oozing tissue deficiencies comprising the step of administering to a patient in need of such treatment a pharmaceutically acceptable amount of the composition of claim 1.

24. A method for the treatment of microbial infections, inflammations, and oozing tissue deficiencies comprising the step of administering to a patient in need of such treatment a pharmaceutically acceptable amount of the composition of claim 9.
